SEQUENCE LISTING (8 sequences)

SEQ ID NO: 1 - DNA, 22 bp, Artificial Sequence
  sense strand of NF-KB oligo probe
agttgaggggactttcccaggc

SEQ ID NO: 2 - DNA, 22 bp, Artificial Sequence
  anti-sense strand of NF-KB oligo probe
gcctgggaaagtcccctcaact

SEQ ID NO: 3 - DNA, 25 bp, Artificial Sequence
  5′ primer for murine TNF-alpha gene
ggcaggtctactttggagtcattgc

SEQ ID NO: 4 - DNA, 25 bp, Artificial Sequence
  3′ primer for murine TNF-alpha gene
acattcgaggctccagtgaattcgg

SEQ ID NO: 5 - DNA, 22 bp, Artificial Sequence
  5′ primer for murine IL-6 gene
ttccatccagttgccttcttgg

SEQ ID NO: 6 - DNA, 23 bp, Artificial Sequence
  3′ primer for murine IL-6 gene
ttctcatttccacgatttcccag

SEQ ID NO: 7 - DNA, 24 bp, Artificial Sequence
  5′ primer for murine 18S rRNA gene
cccggggaggtagtgacgaaaaat

SEQ ID NO: 8 - DNA, 24 bp, Artificial Sequence
  3′ primer for murine 18S rRNA gene
cgcccgctcccaagatccaactac

(19) United States
(12) Patent Application Publication
(10) Pub. No.: US 2006/0223887 A1
(43) Pub. Date: Oct. 5, 2006

(54) Method for treating acute pancreatitis

(76) Inventors: Mitchell P. Fink, Pittsburgh, PA (US); Runkuan Yang, Pittsburgh, PA (US); Russell L. Delude, Pittsburgh, PA (US)

Correspondence Address: HAMILTON, BROOK, SMITH & REYNOLDS, P.C., 530 VIRGINIA ROAD, P.O. BOX 9133, CONCORD, MA, 01742-9133, US

(21) Appl. No.: 11/297,114

(22) Filed: Dec. 8, 2005

Related U.S. Application Data
(63) Continuation of application No. PCT/US03/26475, filed on Aug. 22, 2003.
(60) Provisional application No. 60/476,925, filed on Jun. 9, 2003.

(51) Int. Cl. A61K 31/22; A61K 31/19; A61K 31/16

(52) U.S. Cl. 514/546; 514/557; 514/625

(57) ABSTRACT

Disclosed is a method for treating acute pancreatitis in a subject. The method comprises the step of administering to the subject an effective amount of an ester of an alpha-ketoalkanoic acid or an amide of an alpha-ketoalkanoic acid.

### RELATED APPLICATIONS

This application is a continuation of International Application No. PCT/US2003/26475, filed Aug. 22, 2003, which claims the benefit of U.S. Provisional Application No. 60/476,925, filed Jun. 9, 2003, the entire teachings of which are incorporated herein by reference.

### GOVERNMENT SUPPORT

The invention was supported, in whole or in part, by grants from the Defense Advanced Research Projects Agency (N65236-00-1-5434) and NIH grants GM58484, GM37631 and GM6848 1. The Government has certain rights in the invention.

### BACKGROUND OF THE INVENTION

The pathologic spectrum of acute pancreatitis ranges from relatively mild edematous to severe hemorrhaging or necrotizing pancreatitis, the latter manifesting itself in pancreatic necrosis. While the milder form of acute pancreatitis results in about 1% mortality, necrotizing pancreatitis, which accounts for about one fourth of the cases, has a mortality rate of between 30 to 50%. Still higher mortality rates occur in when the pancreatitis involves infection. Patients with necrotizing pancreatitis suffer a greater risk of serious pancreatic infection and early death with multi-organ failure.

The timing and type of intervention for patients with acute pancreatitis is controversial. Treatment of the milder forms relies mainly on supportive care. Surgical intervention has not been shown to reduce the mortality rates of sterile (non-infected) acute necrotizing pancreatitis, while infected acute necrotizing pancreatitis is considered uniformly fatal without intervention. In either case, necrosectomy and other aggressive surgical procedures remain the standard of care. (Baron, T. H. and Morgan D. E., “Acute Necrotizing Pancreatitis”, The New Engl. J. Med. 340: 1412-1417 (1999).)

Thus, there is an urgent need for new non-invasive methods of preventing and/or ameliorating the effects of acute pancreatitis.

### SUMMARY OF THE INVENTION

It has been found that certain α-keto esters and α-keto amides can be used to ameliorate the effects of acute pancreatitis. For example, administration of Ringer's Ethyl Pyruvate Solution (REPS) to laboratory C57/BL6 mice after inducing an acute pancreatitis improved survival, abrograted bacterial translocation to mesenteric lymph nodes, and significantly lowered circulating levels of alanine aminotransferase (a cellular damage marker). Furthermore, administration of REPS blunted NF-κB DNA binding, down-regulated the expression of TNF-α and IL-6 mRNA in the pancreas, significantly ameliorated systemic microvascular hyperpermeability that was associated with pancreatitis and prevented massive pancreatic necrosis as assessed by staining of fixed sections, compared to control mice administered Ringer's Lactate Solution (Examples 2-10). Accordingly, disclosed herein is a method for treating subjects that have or are at risk for developing acute pancreatitis. The method comprises administering to the subject an effective amount of an ester of an alpha-ketoalkanoic acid or an amide of an alpha-ketoalkanoic.

### BRIEF DESCRIPTION OF THE DRAWINGS

FIG. 1 is a graph showing the effect of Ringer's Lactate Solution (RLS) or Ringer's Ethyl Pyruvate Solution (REPS) on survival of mice with acute pancreatitis.

FIG. 2 is a graph showing the effect of Ringer's Lactate Solution (RLS) and Ringer's Ethyl Pyruvate Solution (REPS) on pancreatic edema in mice with acute pancreatitis. The effect on mice treated with RLS and REPS is compared with untreated control.

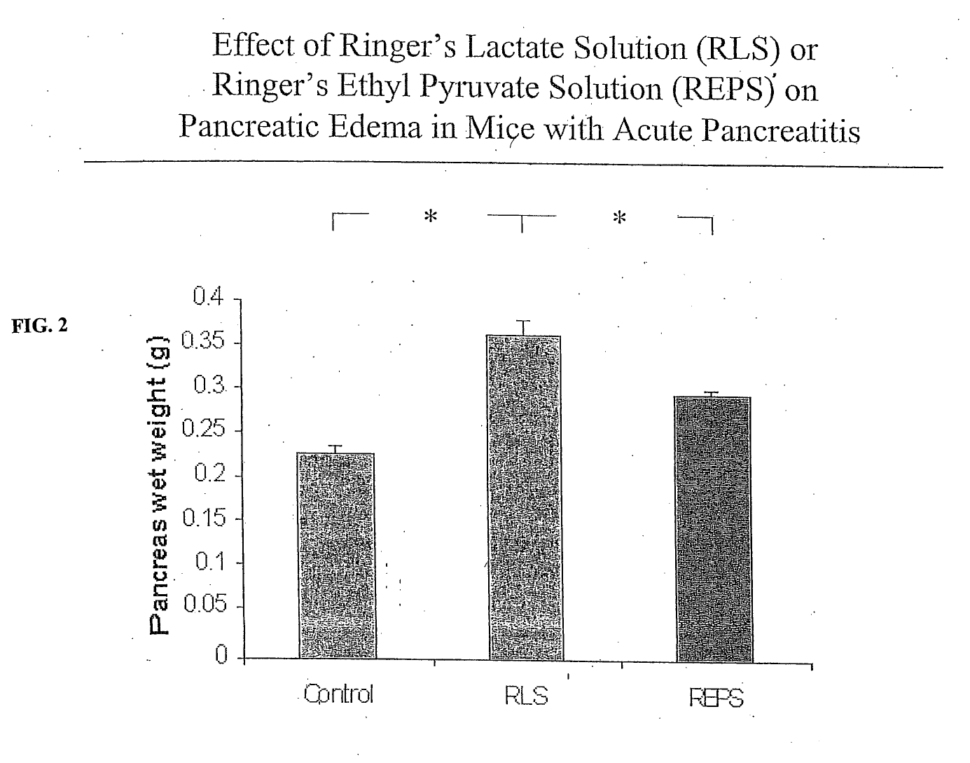

FIG. 3 is a graph showing the effect of Ringer's Lactate Solution (RLS) and Ringer's Ethyl Pyruvate Solution (REPS) on hepatocellular injury in mice with acute pancreatitis. The effect on mice treated with RLS and REPS is compared with untreated control.

FIG. 4 is a graph showing the effect of Ringer's Lactate Solution (RLS) and Ringer's Ethyl Pyruvate Solution (REPS) on expression levels of pancreatic TNF-α and IL-6 mRNA in mice with acute pancreatitis. The effect on mice treated with RLS and REPS is compared with untreated control.

### DETAILED DESCRIPTION OF THE INVENTION

The present invention is a method of treating pancreatitis in a subject by administering an ester of an alpha-ketoalkanoic acid or an amide of an alpha-ketoalkanoic acid dissolved in a physiologically-acceptable vehicle. The disclosed method of treatment is effective in treating the severe forms of acute pancreatitis, including acute necrotizing pancreatitis.

As used herein, the term “pancreatitis” indicates a disease of pancreas whose major causes include excessive alcohol consumption and ductal obstruction (e.g. by gallstones) and whose presentation reflects a continuum of morphologic abnormalities that may include glandular inflammation of pancreas. In the acute stage, this ranges from mild disease (edematous pancreatitis) to the severe form (hemorrhagic or necrotizing pancreatitis). The former is characterized by exudation of neutrophils and interstitial edema with apparent preservation of parenchymal elements, the latter by coagulation necrosis of the gland and surrounding fatty tissue, resulting in loss of structural integrity, and, possibly, bleeding. Severe acute pancreatitis is usually a result of pancreatic glandular necrosis. The morbidity and mortality associated with acute pancreatitis are substantially higher when necrosis is infected (i.e., “infected acute pancreatitis”). Acute pancreatitis usually has a rapid onset manifested by upper abdominal pain, vomiting, fever, tachycardia, leukocytosis, and elevated serum levels of pancreatic enzymes. The disclosed method can be used to treat all of these forms of pancreatitis.

The method of the present invention can be used to treat acute pancreatitis at the time of onset, and is also suited for prophylactic treatment of acute pancreatitis. “Prophylactic treatment” refers to treatment before onset of the disease to prevent, inhibit or reduce the occurrence of acute pancreatitis. For example, a subject at risk for acute pancreatitis, such as a subject with mild or chronic pancreatitis or a subject about to undergo a procedure associated with development of acute pancreatitis as a complication, such as endoscopic retrograde cholangiopancreatography, can be prophylactically treated according to the method of the present invention prior to the onset of acute pancreatitis. A subject at risk for pancreatitis” can also be a subject who is being treated with a drug that can cause pancreatits (see below). The disclosed method can be used in combination with such treatments.

The terms “therapeutic” and “treatment” as used herein, refer to ameliorating symptoms associated with a disease or condition, including preventing, inhibiting or delaying the onset of the disease symptoms, and/or lessening the severity, duration or frequency of symptoms of the disease.

A “subject” is preferably a human patient, but can also be a companion animal (e.g., dog, cat and the like), a farm animal (e.g., horse, cow, sheep, and the like) or laboratory animal (e.g., rat, mouse, guinea pig, and the like).

The major causes of acute pancreatitis are alcohol abuse and gallstones, which together account for approximately 75% of all cases. Other causes include drugs such as imuran, DDI and pentamidine, infections such as CMV, hypertriglyceridemia, hypercalcemia and hypotension. Pancreatitis can also have mechanical causes such as ductal obstructions which commonly occur in patients with carcinoma of the pancreas, post-operative and post endoscopic retrograde cholangiopancreatography (post-ERCP) as well as trauma-related causes.

Acute pancreatitis can be induced by alcohol ingestion, biliary tract disease (gallstones), postoperative state (after abdominal or nonabdominal operation), endoscopic retrograde cholangiopancreatography (ERCP), especially manometric studies of sphincter of Oddi, trauma (especially blunt abdominal type), or metabolic causes such as hypertriglyceridemia, apolipoprotein CII deficiency syndrome, hypercalcemia (e.g., hyperparathyroidism), renal failure drug-induced or as a result of renal transplantation, or acute fatty liver of pregnancy. The acute pancreatitis can be a hereditary pancreatitis or can be caused by infections such as mumps, viral hepatitis, other viral infections including coxsackievirus, echovirus, and cytomegalovirus, ascariasis, or infections with Mycoplasma, Campylobacter, Mycobacterium avium complex. Pancreatitis can also be induced by medicaments or drugs such as azathioprine, 6-mercaptopurine, sulfonamides, furosemide, thiazide diuretics, estrogens (oral contraceptives), tetracycline, pentamidine, valproic acid, dideoxyinosine, acetaminophen, nitrofurantoin, erythromycin, methyldopa, salicylates, metronidazole, nonsteroidal anti-inflammatory drugs, or angiotensin-converting enzyme (ACE) inhibitors. The method of the present invention can also be used to treat pancreatitis caused by ischemic-hypoperfusion state (after cardiac surgery), atherosclerotic emboli, systemic lupus erythematosus, necrotizing angiitis, trombotic thrombocytopenic purpura, penetrating peptic ulcer, obstruction of the ampulla of Vater, regional enteritis, duodenal diverticulum, or pancreas divisum.

In one aspect, the therapeutic agent used in the method disclosed herein is an effective amount of an ester of an alpha-ketoalkanoic acid, for example, a C₃—C₈ straight-chained or branched alpha-ketoalkanoic acid. Examples include alpha-keto-butyrate, alpha-ketopentanoate, alpha-keto-3-methyl-butyrate, alpha-keto-4-methyl-pentanoate or alpha-keto-hexanoate. Pyruvate is preferred. A variety of groups such as alkyl, aralkyl, alkoxyalkyl, carbalkoxyalkyl or acetoxyalkyl are suitable for the ester position of the molecule. Specific examples include ethyl, propyl, butyl, carbmethoxymethyl (—CH₂COOCH₃), carbethoxymethyl (—CH₂COOCH₂CH₃), acetoxymethyl (—CH₂OC(O)CH₃), carbmethoxyethyl (—CH₂CH2COOCH₃), carbethoxyethyl (—CH₂CH₂COOCH₂CH₃), methoxymethyl (—CH₂OCH₃) and ethoxymethyl (—CH₂OCH₂CH₃). Ethyl esters are preferred. Thiolesters (e.g., wherein the thiol portion is cysteine or homocysteine) and glyceryl esters (e.g., wherein one or more of the alcohol groups on glycerol are replaced with an α-ketoalkanoate group) are also included. Other groups suitable for esterification of alpha-ketoalkanoic acids include: 1) dihydroxyacetone esters of formula: 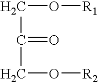
wherein R₁ is an α-ketoalkanoate group such as pyruvyl and R₂ is H, an α-ketoalkanoate group such as pyruvyl or a C1—C3 acyl group such as acetyl or propionyl; and 2) monosaccharide esters such as ribosyl and glucosyl esters: 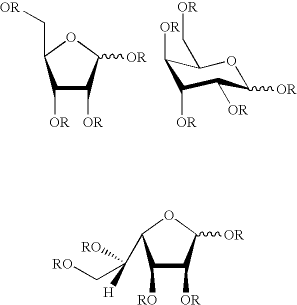
wherein each R is independently H, an α-ketoalkanoate group such as pyruvyl or a C1—C3 acyl group such as acetyl or propionyl, provided that at least one R is an α-ketoalkanoate group.

Examples of alpha-ketoalkanoic acid esters suitable for use in the disclosed method include ethyl pyruvate, propyl pyruvate, carbmethoxymethyl pyruvate, acetoxymethyl pyruvate, carbethoxymethymethyl pyruvate, ethoxymethyl pyruvate, ethyl alpha-keto-butyrate, ethyl alpha-keto-pentanoate, ethyl alpha-keto-3-methyl-butyrate, ethyl alpha-keto-4-methyl-pentanoate, or ethyl alpha-keto-hexanoate. Ethyl pyruvate is a preferred alpha-ketoalkanoic acid ester.

In yet another aspect, the therapeutic agent used in the method disclosed herein is an effective amount of an amide of an alpha-ketoalkanoic acid.

Suitable amides of alpha-ketoalkanoic acids for use in the method of the present inventions include compounds having the following structural formula:

RCOCONR1R2. R is an alkyl group; R1 and R2 are independently —H, alkyl, aralkyl, alkoxyalkyl, carbalkoxyalkyl or —CHR3COOH (i.e. an “amino acid amide of an alpha-ketoalkanoic acid”); and R3 is the side chain of a naturally occurring amino acid. Preferably, the amide of an alpha-ketoalkanoic acids is a pyruvamide.

Suitable alkyl groups include C₁—C₈ straight chained or branched alkyl group, preferably C₁—C₆ straight chained alkyl groups.

Suitable aryl groups include carbocyclic (e.g., phenyl and naphthyl) and heterocyclic (e.g., furanyl and thiophenyl) aromatic groups, preferably phenyl.

An alkoxy group is —OR4, wherein R4 is an alkyl group, as defined above. An alkoxyalkyl group is an alkyl group substituted with —OR4.

An aralkyl group is —XY, wherein X is an alkyl group and Y is an aryl group, both as defined above.

A carboxyalkyl group is an alkyl group substituted with —COOH. A carbalkoxyalkyl group is an alkyl group substituted with —C(O)OR, wherein R is an alkyl group, as defeind above.

An acyl group is —C(O)—R, wherein R is an alkyl group, as defined above.

An acetoxy alkyl group is an alkyl group substituted with —O—C(O)—R, wherein R is an alkyl group, as defined above.

Formulation of a therapeutic agent to be administered will vary according to the route of administration selected (e.g., solution, emulsion, capsule). An appropriate composition comprising the agent to be administered can be prepared in a physiologically or pharmaceutically acceptable vehicle or carrier. A physiologically or pharmaceutically acceptable carrier for the composition used in the method of the present invention can be any carrier vehicle generally recognized as safe for administering a therapeutic agent to a mammal, e.g., a buffer solution for infusion or bolus injection, a tablet for oral administration or in gel, micelle or liposome form for on-site delivery. A preferred buffer solution is water or isotonic or hypertonic saline buffered with bicarbonate, phosphate, lactate or citrate at 0.1 M to 0.2 M. Alternatively, the therapeutic agent is administered in a plasma extender, microcolloid or microcrystalline solution. One preferred carrier is Ringer's isotonic saline solution comprising from about 105 mM to 110 mM NaCl, from about 3.8 mM to about 4.2 mM KCl , and from about 2.5 to 2.9 mM CaCl₂. More preferably, the carrier is Ringer's Lactate solution comprising from about 105 mM to 110 mM NaCl, from about 3.8 mM to about 4.2 mM KCl , and from about 2.5 to 2.9 mM CaCl₂, and from about 25 mM to about 30 mM of sodium lactate. Preferably, acidity of the formulation is adjusted to a pH range of about 4 to about 8, even more preferably to a pH value of about 5 to about 7. Other carriers for the compounds of the present invention include isotonic salt solutions buffered with citrate, for example, approximately 100 mM to 200 mM citrate.

A preferred concentration range of the therapeutic agent is from about 0.1 to about 10% by weight. In a particularly preferred aspect, the pharmaceutical composition comprises approximately 10 mg/ml of ethyl pyruvate. A preferred example of the formulation used for treating pancreatitis comprises 2% to 3% ethyl pyruvate by weight, approximately 100 mM citrate buffer (or about 25 mM to about 30 mM of sodium lactate), about 4 mM KCl and, optionally, 2.7 mM CaCl₂. The formulation administered for the treatment of pancreatitis can be formed by admixing components of a two part formulation, one part containing, for example, ethyl pyruvate (neat), and the second part consisting of the remaining components of a desired aqueous formulation, for example, those reagents described above.

The pharmaceutical compositions used in the method of the present invention can optionally include an enolization agent when the therapeutic agent is an α-keto ester. The enolization agent and an α-keto ester are contained in a physiologically acceptable carrier. An “enolization agent” is a chemical agent, which induces and stabilizes the enol resonance form of an alpha-keto ester at or around physiological pH (e.g., between about 4.0 to about 8.0, more preferably between about 4.5 to about 6.5). Enolization agents include a cationic material, preferably a divalent cation such as calcium or magnesium or, for example, a cationic amino acid such ornithine or lysine. Divalent cations are introduced into the pharmaceutical formulation as a salt, e.g., as calcium chloride or magnesium chloride. The enolization agent in the composition of the invention is at an appropriate concentration to induce enolization of the alpha-keto functionality of the amount of active ester agent in the administered composition, e.g., from 0.0 to 4.0 molar equivalents relative to the ester.

The precise dose to be employed in the formulation of a therapeutic agent will depend on the route of administration, and the seriousness of the conditions, and should be decided according to the judgment of a practitioner and each patient's circumstances. Effective doses may be extrapolated from dose-response curves derived from in vitro or animal model test systems.

According to the method, one or more ester of an alpha-ketoalkanoic acid or amide of alpha-ketoalkanoic acid can be administered to a subject by an appropriate route, either alone or in combination with another drug. An effective amount of an alpha-ketoalkanoic acid or physiologically-acceptable salt thereof, an ester of an alpha-ketoalkanoic acid, or an amide of alpha-ketoalkanoic acid is administered. An effective amount is an amount sufficient to achieve the desired therapeutic or prophylactic effect, under the conditions of administration, such as an amount sufficient for treating (therapeutically or prophylactically) acute pancreatitis.

The therapeutic compositions of the invention can be administered through a variety of routes, for example, oral, dietary, topical, intravenous, intramuscular, or by inhalation (e.g., intrabronchial, intranasal or oral inhalation, intranasal drops) routes of administration, depending on the agent and disease or condition to be treated, using routine methods in physiologically-acceptable inert carrier substances. Other suitable methods of administration can also include rechargeable or biodegradable devices, and slow release polymeric devices. For example, the therapeutic compositions can be administered in a sustained release formulation using a biodegradable biocompatible polymer, or by on-site delivery using micelles, gels, liposomes, or a buffer solution. Preferably, the pharmaceutical composition is administered as an infusate at a concentration of, e.g., 10 mM to 200 mM, preferably 20 mM to 90 mM of the active agent, at a rate of 1 mg/kg body weight/day to 200 mg/kg body weight/day, in a buffer solution as described herein. More preferably, the pharmaceutical composition is administered as an infusate at a concentration of about 28 mM of the active agent at a dose of 100 mg/kg body weight/day to 150 mg/kg body weight/day of alpha-ketoalkanoic acid, in a buffer solution. In bolus form, the active agent can be administered at a similar dosage, e.g., 1 mg/kg body weight/day to 200 mg/kg body weight/day of active agent, where the dosage is divided into aliquots and delivered 1 to 4 times daily (for a total dosage of 1 mg/kg body weight/day to 200 mg/kg body weight/day), with the concentration of the active agent adjusted accordingly. Optimal dosage and modes of administration can readily be determined by conventional protocols.

The α-keto acids and α-keto esters disclosed herein for the treatment of pancreatitis can be administered as a monotherapy (i.e., alone as the sole therapeutic agent being used to treat the pancreatitis) or in combination with other pharmaceutical agents, e.g., anti-microbials, anti-inflammatory agents, analgesics, anti-viral agents, anti-fungals, anti-histamines and the like.

Examples of suitable anti-microbial agents include sulfa drugs, pencillins (e.g., Benzyl penicillin, P-hydroxybenzyl penicillin, 2-pentenyl penicillin, N-heptyl penicillin, phenoxymethyl penicillin, Phenethicillin, Methicillin, Oxacillin, Cloxacillin, Dicloxacillin, Flucloxacillino, Nafcillin, Ampicillin, Amoxicillin, Cyclacillin, Carbenicillin, Ticarcillin, Piperacillin, Azlocillin, Meczlocillin, Mecillinam, Amdinocillin), Cephalosporin and derivatives thereof (e.g, Cephalothin, Cephapirin, Cephacetrile, Cephazolin, Caphalexin, Cephandine, Cefadroxil, Cefamandol, Cefuroxime, Ceforanide, Cefoxitin, Cefotetan, Cefaclor, Cefotaxime, Ceftizoxime, Ceftrioxone, Ceftazidime, Moxalactam, Cefoperazone, Cefixime, Ceftibuten and Cefprozil), Oxolinic Acid, Amifloxacin, Temafloxacin, Nalidixic Acid, Piromidic Acid, Ciprofloxacin, Cinoxacin, Norfloxacin, Perfloxacin, Rosaxacin, Ofloxacin, Enoxacin, Pipemidic Acid, Sulbactam, Clavulinic Acid, β-Bromopenicillanic Acid, β-Chloropenicillanic Acid, 6-Acetylmethylene-Penicillanic Acid, Cephoxazole, Sultampicillin, Formaldehyde Hudrate Ester of Adinocillin and Sulbactam, Tazobactam, Aztreonam, Sulfazethin, Isosulfazethin, Norcardicins, m-Carboxyphenyl Phenylacetamidomethylphosphonate, Chlortetracycline, Oxytetracyline, Tetracycline, Demeclocycline, Doxycycline, Methacycline and Minocycline.

Examples of suitable anti-inflammatory agents include examples of suitable NSAIDs include aminoarylcarboxylic acid derivatives (e.g., Enfenamic Acid, Etofenamate, Flufenamic Acid, Isonixin, Meclofenamic Acid, Niflumic Acid, Talniflumate, Terofenamate and Tolfenamic Acid), arylacetic acid derivatives (e.g., Acematicin, Alclofenac, Amfenac, Bufexamac, Caprofen, Cinmetacin, Clopirac, Diclofenac, Diclofenac Sodium, Etodolac, Felbinac, Fenclofenac, Fenclorac, Fenclozic Acid, Fenoprofen, Fentiazac, Flubiprofen, Glucametacin, Ibufenac, Ibuprofen, Indomethacin, Isofezolac, Isoxepac, Ketoprofen, Lonazolac, Metiazinic Acid, Naproxen, Oxametacine, Proglumrtacin, Sulindac, Tenidap, Tiramide, Tolectin, Tolmetin, Zomax and Zomepirac), arylbutyric acid ferivatives (e.g., Bumadizon, Butibufen, Fenbufen and Xenbucin) arylcarboxylic acids (e.g., Clidanac, Ketorolac and Tinoridine), arylproprionic acid derivatives (e.g., Alminoprofen, Benoxaprofen, Bucloxic Acid, Carprofen, Fenoprofen, Flunoxaprofen, Flurbiprofen, Ibuprofen, Ibuproxam, Indoprofen, Ketoprofen, Loxoprofen, Miroprofen, Naproxen, Oxaprozin, Piketoprofen, Piroprofen, Pranoprofen, Protinizinic Acid, Suprofen and Tiaprofenic Acid), pyrazoles (e.g., Difenamizole and Epirizole), pyrazolones (e.g., Apazone, Benzpiperylon, Feprazone, Mofebutazone, Morazone, Oxyphenbutazone, Phenylbutazone, Pipebuzone, Propyphenazone, Ramifenazone, Suxibuzone and Thiazolinobutazone), salicyclic acid derivatives (e.g., Acetaminosalol, 5-Aminosalicylic Acid, Aspirin, Benorylate, Biphenyl Aspirin, Bromosaligenin, Calcium Acetylsalicylate, Diflunisal, Etersalate, Fendosal, Flufenisal, Gentisic Acid, Glycol Salicylate, Imidazole Salicylate, Lysine Acetylsalicylate, Mesalamine, Morpholine Salicylate, 1 -Naphthyl Sallicylate, Olsalazine, Parsalmide, Phenyl Acetylsalicylate, Phenyl Salicylate, 2-Phosphonoxybenzoic Acid, Salacetamide, Salicylamide O-Acetic Acid, Salicylic Acid, Salicyloyl Salicylic Acid, Salicylsulfuric Acid, Salsalate and Sulfasalazine), thiazinecarboxamides (e.g., Droxicam, Isoxicam, Piroxicam and Tenoxicam), ε-Acetamidocaproic Acid, S-Adenosylmethionine, 3-Amino-4-hydroxybutyric Acid, Amixetrine, Bendazac, Benzydamine, Bucolome, Difenpiramide, Ditazol, Emorfazone, Guaiazulene, Ketorolac, Meclofenamic Acid, Mefenamic Acid, Nabumetone, Nimesulide, Orgotein, Oxaceprol, Paranyline, Perisoxal, Pifoxime, Piroxicam, Proquazone and Tenidap.

Examples of suitable analgesics include an opioid (e.g. morphine), a COX-2 inhibitor (e.g., Rofecoxib, Valdecoxib and Celecoxib), salicylates (e.g., ASPIRIN, choline magnesium trisalicylate, salsalate, difunisal and sodium salicylate), propionic acid derivatives (e.g., fenoprofen calcium, ibuprofen, ketoprofen, naproxen and naproxen sodium, indoleacetic acid derivatives (e.g., indomethacin, sulfindac, etodalac and tolmetin), fenamates (e.g., mefenamic acid and meclofenamate), benzothiazine derivatives or oxicams (e.g., mobic or piroxicam) or pyrrolacetic acid (e.g., ketorolac).

Examples of suitable anti-viral agents include inferno gamma, ribavirin, fialuridine, acyclovir, ganciclovir, penciclovir, famciclovir, PMEA, bis-POM PMEA, lamivudine, cytallene, oxetanocins, carbocyclic oxetaoncins, foscarnet, phyllanthus amarus, N-acety-L-cysteine, destruxin B, hypericin, aucubin and N-butyldeoxynojirimycin.

Examples of suitable anti-fungals include amphotericin B, nystatin, itraconazole, fluconazole, ketoconazole, miconazole, flucytosine and dapsone.

### EXEMPLIFICATION

The present invention will now be illustrated by the following Examples, which are not intended to be limiting in any way.

Example 1 Induction of Murine Acute Necrotizing Pancreatitis in a Mouse Model

Acute necrotizing pancreatitis was induced by feeding C57Bl/6 mice a choline-deficient diet supplemented with 0.5% ethionine for one day. Subsequently the mice were challenged with seven hourly doses of cerulein (0.05 mg/kg intraperitoneally) followed by an intraperitoneal (i.p.) injection of Escherichia coli lipopolysaccharide (LPS) (4 mg/kg). Six hours before and 1 hour after the LPS injection, the mice were randomized to receive doses of either REPS (40 mg/kg, i.p.) or a similar volume of RLS. Mice in the control group were injected with equivalent volumes of phosphate buffered saline (pH 7.40) instead of cerulein or LPS.

Example 2 Treatment With REPS But Not RLS Improved Survival in a Mouse Model

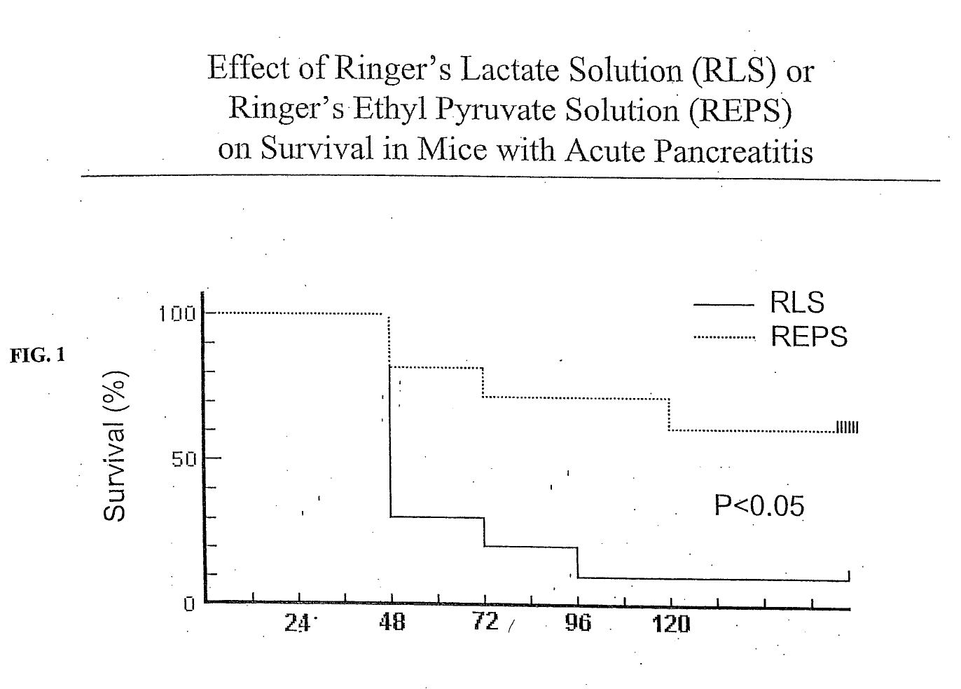

Mice with murine acute necrotizing pancreatitis induced as in Example 1 were subjected to i.p. injections of RLS or REPS (equivalent to 50 mg/kg ethyl pyruvate) 2 hours after injection of LPS. Dosing with RLS or REPS was repeated every 6 h for 48 h. As demonstrated in FIG. 1, a 7-day survival in control group was 100% ( 10/10), in RLS group is 10% ( 1/10); while in REPS group is 60% ( 6/10).

Example 3 Treatment With REPS But Not RLS Significantly Ameliorated Damage to Pancreas as Evidenced by Systemic Microvascular Hyperpermeability in a Mouse Model

In order to assess acute lung injury associated with pancreatitis, mice were injected intravenously with FITC-albumin. Bronchoalveolar lavage was then performed 120 minutes later.

Mice with murine acute necrotizing pancreatitis induced as in Example 1 were subjected to i.p. injections of RLS or REPS (equivalent to 50 mg/kg of ethyl pyruvate) 1 hour prior to starting injections of cerulein. A second dose was injected 6 h later. A control group was injected with PBS. The animals were also infused via the tail vein with fluorescein isothiocyanate (FITC)-albumin (5 mg/kg in 0.3 mL PBS) just before being injected with endotoxin.

All animals were sacrificed and the trachea was exposed and the lungs were lavaged three times with 1 ml of PBS and blood was collected by cardiac puncture. The bronchoalveolar lavage (BALF) fluid was pooled and serum was collected. FITC-albumin concentrations win in BALF and serum were determined fluorometrically (excitation=494 nm; emission=520 nm). The BALF/serum fluorescence ration was calculated and used as a measure of damage to pulmonary alveolar endothelial/epithelial integrity.

The BALF/serum FITC-albumin ratio was more than five-fold greater in the RLS group as compared to the control group. Treatment with ethyl pyruvate significantly ameliorated acute lung injury (REPS versus RLS comparison), although the BALF/serum FITC-albumin ratio was still significantly greater in the REPS group than in the control group.

Example 4 Treatment With REPS But Not RLS Ameliorated Pancreatic Edema in a Mouse Model

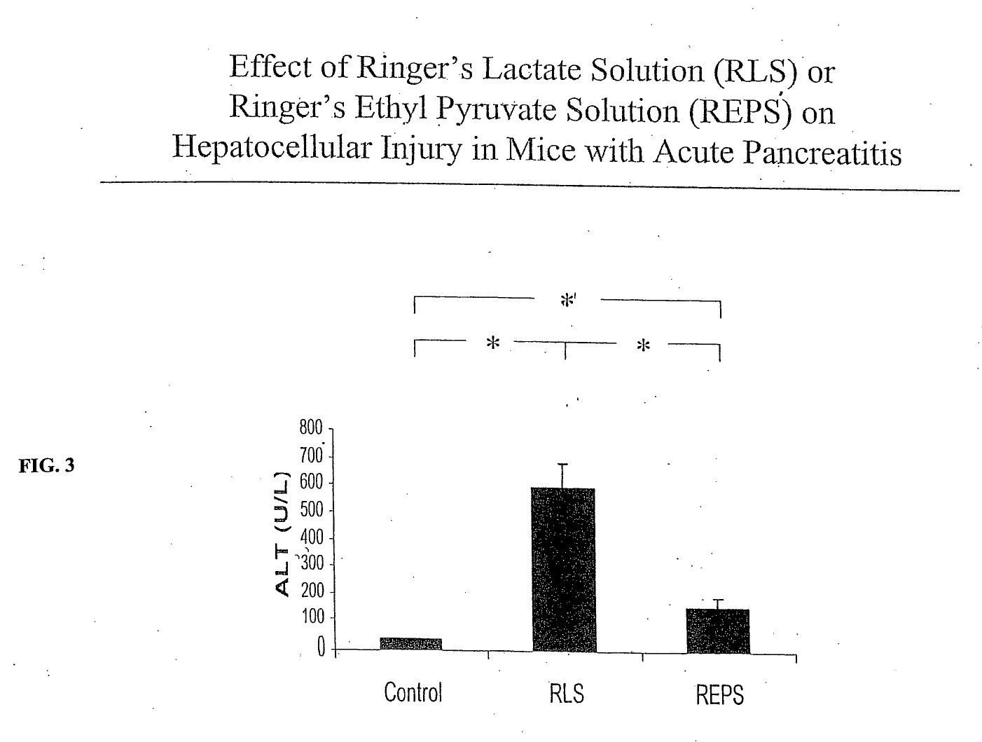

Mice with murine acute necrotizing pancreatitis induced as in Example 1 were subjected to i.p. injections of RLS or REPS (equivalent to 50 mg/kg of ethyl pyruvate) 1 hour prior to starting injections of cerulein. A second dose was injected 6 h later. All animals were sacrificed for obtaining tissue and blood samples 10 h after the first cerulein injection. Pancreata were harvested from each animal and weighed to determine the extent of edema. As indicated in FIG. 3, pancreatic edema was alleviated in a group of mice administered REPS but not RLS.

Example 5 Treatment With REPS But Not RLS Alleviates Inflammatory Response as Evidenced by Inhibitory Effect on NF-kB Activation in Pancreas in a Mouse Model

Mice with murine acute necrotizing pancreatitis induced as in Example 1 were subjected to i.p. injections of RLS or REPS (equivalent to 50 mg/kg of ethyl pyruvate) 1 hour prior to starting injections of cerulein. A second dose was injected 6 hours later. All animals were sacrificed for obtaining tissue and blood samples 10 hours after the first cerulein injection.

To prepare nuclear extracts, pancreatic tissue samples were homogenized with T-PER™ (Pierce, Rockford, Ill.), using a 1:20 ratio of tissue to the sample preparation reagent, as directed by the manufacturer's instructions. The samples were centrifuged at 10,000 g for 5 min to pellet tissue debris. The supernatant was collected and frozen at −80° C. Nuclear protein concentration was determined using a commercially available Bradford assay (Bio-Rad, Hercules, Calif.).

The EMSA for NF-κB nuclear binding was performed using a duplex oligonucleotide probe based on the NF-κB binding site upstream of the murine iNOS promoter as previously described (Yang R, et al., “Ethyl pyruvate modulates inflammatory gene expression in mice subjected to hemorrhagic shock”, Am. J. Physiol. Gastrointest. Liver Physiol. 283: G212-G22 (2002)). The sequence of the double-stranded NF-κB oligonucleotide was as follows: sense: 5′-AGT TGA GGG GAC TTT CCC AGG C-3′ (SEQ ID NO: 1); antisense: 3′-TCA ACT CCC CTG AAA GGG TCC G-5′ (SEQ ID NO: 2) (Promega; Madison, Wis.). The oligonucleotides were end-labeled with γ−³²P adenosine triphosphate (New England Nuclear; Boston, Mass.) using T4 polynucleotide kinase (Promega; Madison, Wis.). 6 μg of nuclear protein was incubated at room temperature with γ−³²P-labeled NF-κB probe in 4 μl of 5X binding buffer (65 mM HEPES, 325 mM NaCl, 5 mM DTT, 0.7 mM EDTA, 40% glycerol, pH=8.0) in the presence of 2 μg of polyDI-DC for 20 min, the total volume of the binding reaction mixture being 20 μL. The binding reaction mixture was electrophoresed on 4% nondenaturing polyacrylamide electrophoresis gels. After electrophoresis, the gels were dried and exposed to Kodak (Rochester, N.Y.) X-Omat film at −80° C. The specificity of the binding reaction has been previously verified by carrying out appropriate cold-competition and super-shift assays.

To determine the specificity of binding reactions, “cold competition” studies were carried out using a 100-fold molar excess of either unlabeled NF-B duplex oligonucleotide (specific competition) or an irrelevant oligonucleotide probe. For the latter, we used a probe containing the hypoxia inducible factor (HIF)-1 binding sequence from the human erythropoeitin 3′ enhancer. Supershift assays were performed by incubating nuclear extracts with 2 μL of anti-p65 and anti-p50 antibodies (Santa Cruz Biotechnology; Santa Cruz, Calif.) for 1 h prior to the addition of radiolabeled probe. The binding reaction mixture was electrophoresed on 4% PAGE gels. After electrophoresis, the gels were dried and exposed to XAR-5 film (Kodak; Rochester, N.Y.) at −80° C. for overnight using an intensifying screen. To confirm the identity of the activated protein-DNA complex, binding assays were carried out with samples that were pre-incubated with specific antibodies directed against p50 and p65.

The results of the EMSA for NF-κB nuclear binding indicated that there was a marked increase in activation of NF-κB following induction of acute pancreatitis. However, treatment of mice with REPS but not RLS after induction down-regulated NF-κB DNA binding.

Example 6 Treatment With REPS But Not RLS in a Mouse Model Alleviates Damage to the Liver as Evidenced by Significantly Lower Circulating Levels of Alanine Aminotransferase

Mice with murine acute necrotizing pancreatitis induced as in Example 1 were subjected to i.p. injections of RLS or REPS (equivalent to 50 mg/kg of ethyl pyruvate) 1 hour prior to starting injections of cerulein. A second dose was injected 6 hours later. All animals were sacrificed for obtaining tissue and blood samples 10 hours after the first cerulein injection.

200 μL of blood was obtained by cardiac puncture and placed in a 0.5 ml centrifugation tube on ice. The samples were centrifuged at 5,000 g for 3 min. The serum was collected and assayed for Alanine Amino Transferase (ALT) using an automated assay system.

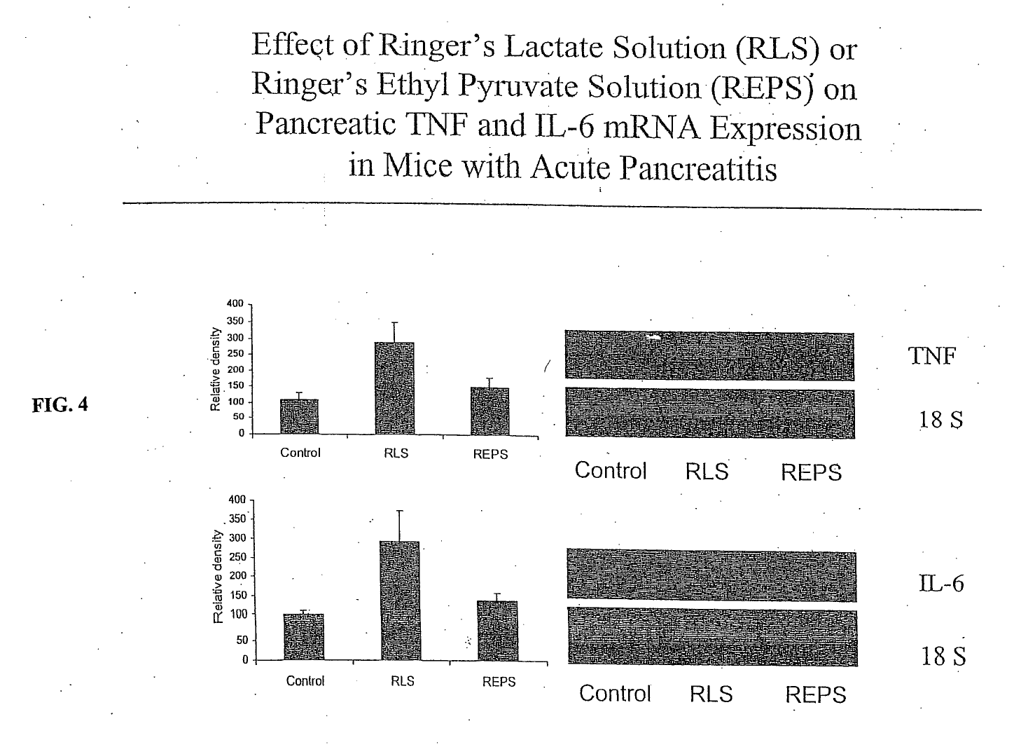

As shown in FIG. 4, the mean plasma ALT concentration at 4 hours after injection of LPS was significantly greater in the RLS group than in the control group. However, the mean circulating level of this biochemical marker of cellular injury was significantly lower in the REPS group than in the RLS group. There was no statistical difference between the control and REPS groups.

Example 7 Treatment With REPS But Not RLS Inhibits Inflammatory Response as Measured by Expression of TNF-α and IL-6 mRNA in the Pancreas

Mice with murine acute necrotizing pancreatitis induced as in Example 1 were subjected to i.p. injections of RLS or REPS (equivalent to 50 mg/kg of ethyl pyruvate) one hour prior to starting injections of cerulein. A second dose was injected 6 hours later. All animals were sacrificed for obtaining tissue and blood samples 10 hours after the first cerulein injection.

Total RNA was extracted from harvested tissues with chloroform and TRI Reagent (Molecular Research Center, Cincinnati, Ohio) exactly as directed by the manufacturer. The total RNA was treated with DNAFree (Ambion, Houston, Tex.) as instructed by the manufacturer using 10 units of DNase I/10 μg RNA. Two μg of total RNA was reverse transcribed in a 40 μl reaction volume containing 0.5 μg of oligo(dT)₁₅ (Promega), 1 mM of each dNTP, 15 U AMV reverse transcriptase (Promega), and 1 U/μL of recombinant RNasin ribonuclease inhibitor (Promega) in 5 mM MgCl₂, 10 mM Tris-HCl, 50 mM KCL, 0.1% Triton X-100 (pH=8.0). The reaction mixtures were preincubated at 21° C. for 10 min prior to DNA synthesis. The RT reactions were carried out for 50 min at 42° C. and were heated to 95° C. for 5 min to terminate the reaction. Reaction mixtures (50 μL) for PCR were assembled using 5 μL of cDNA template, 10 units AdvanTaq Plus DNA Polymerase (Clontech, Palo Alto, Calif.), 200 μM of each dNTP, 1.5 mM MgCl₂ and 1.0 μM of each primer in 1x AdvanTaq Plus PCR buffer. PCR reactions were performed using a Model 480 thermocycler (Perkin Elmer, Norwalk, Conn.). Amplication was initiated with 5 min of denaturation at 94° C. The PCR conditions for amplifying cDNA for TNF, IL-6 were as follows: denaturation at 94° C. for 45 s, annealing at 61° C. for 45 s, and polymerization at 72° C. for 45 s. In order to ensure that amplification was in the linear range, the optimal number of cycles were empirically identified as 33 and 35 for TNF and IL-6, respectively. After the last cycle of amplification, the samples were incubated at 72° C. for 10 min and then held at 4° C. The 5′ and 3′ primers for TNF (Invitrogen Corp.; Carlsbad, Calif.) were GGC AGG TCT ACT TTG GAG TCA TTG C (SEQ ID NO: 3) and ACA TTC GAG GCT CCA GTG AAT TCG G (SEQ ID NO: 4), respectively; the expected product length was 307 bp. The 5′ and 3′ primers for IL-6 were TTC CAT CCA GTT GCC TTC TTG G (SEQ ID NO: 5) and TTC TCA TTT CCA CGA TTT CCC AG (SEQ ID NO: 6), respectively; the expected product length was 174 bp. 18S ribosomal RNA was amplified to verify equal loading. For this reaction, the 5′ and 3′ primers CCC GGG GAG GTA GTG ACG AAA AAT (SEQ ID NO: 7) and CGC CCG CTC CCA AGA TCC AAC TAC (SEQ ID NO: 8), respectively; the expected product length was 209 bp. Ten μl of each PCR reaction product was electrophoresed on a 2% agarose gel, scanned using a NucleoVision imaging workstation (NucleoTech, San Mateo, Calif.), and quantified using GelExpert™ release 3.5.

Using semi-quantitative RT-PCR, we showed that pancreatic TNF-α mRNA and IL-6 mRNA expression was markedly increased upon inducing the acute pancreatitis. As presented on FIG. 5, in mice treated with REPS but not RLS, upregulation of pancreatic TNF-α mRNA and IL-6 mRNA expression was not observed.

Example 8 Treatment with REPS But Not RLS Prevents Pancreatic Cellular Damage as Evidenced by Histological Examination

Formalin-fixed hepatic tissue was sectioned, stained with hematoxylin and eosin, and examined using light microscopy at 600 magnification.

Following treatment with RLS, pancreatic acini showed evidence of diffuse necrosis. Nuclei of acinar cells were lysed. There were scattered neutrophils infiltrating the parenchyma and the islets are shrunken. In contrast to RLS, the group treated with REPS showed only minimal evidence of necrosis and no evidence of neutrophilic infiltration.

While this invention has been particularly shown and described with references to preferred embodiments thereof, it will be understood by those skilled in the art that various changes in form and details may be made therein without departing from the scope of the invention encompassed by the appended claims.

What is claimed is:

1. A method of treating acute pancreatitis in a subject, said method comprising administering to said subject an effective amount of an ester of an alpha-ketoalkanoic acid, an amide of an alpha-ketoalkanoic acid or an alpha-ketoalkanoic acid or salt thereof.

2. The method of claim 1 wherein said acute pancreatitis is acute edematous pancreatitis.

3. The method of claim 1 wherein said acute pancreatitis is acute hemorrhaging pancreatitis.

4. The method of claim 1 wherein said pancreatitis is acute necrotizing pancreatitis.

5. The method of claim 1 wherein said pancreatitis is infected acute pancreatitis.

6. The method of claim 1 wherein said subject is treated prophylactically for acute pancreatitis.

7. The method of claim 1, wherein the subject is administered an ester of an alpha-alkanoic acid and wherein the alpha-ketoalkanoic acid ester is a C₃—C₈ straight-chained or branched alpha-ketoalkanoic acid ester.

8. The method of claim 7, wherein said ester of an alpha-ketoalkanoic acid is an alkyl, aralkyl, alkoxyalkyl or carbalkoxyalkyl ester.

9. The method of claim 7, wherein said ester of an alpha-ketoalkanoic acid is an ethyl ester.

10. The method of claim 7, wherein said ester of an alpha-ketoalkanoic acid is an ester of pyruvic acid.

11. The method of claim 7 wherein said ester of alpha-ketoalkanoic acid is contained in Ringer's isotonic saline.

12. The method of claim 7, wherein said ester of an alpha-ketoalkanoic acid is contained in a physiologically-acceptable carrier, which further comprises lactate.

13. The method of claim 12 wherein said physiologically-acceptable carrier further comprises a physiologically-acceptable enolization agent.

14. The method of claim 12, wherein said physiologically-acceptable carrier is Ringer's isotonic saline comprising potassium ion and/or sodium ion.

15. The method of claim 7, wherein said alpha-ketoalkanoic acid ester is a glyceryl ester.

16. The method of claim 7, wherein said alpha-ketoalkanoic acid ester is a ribosyl ester of the following formula: 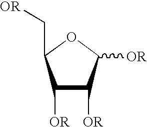
wherein each R is independently H, an alpha-ketoalkanoate group or a C1—C3 acyl and at least one R is an alpha-ketoalkanoate group.

17. The method of claim 7, wherein said alpha-ketoalkanoic acid ester is a glucosyl ester represented by formulae (I) or (II): 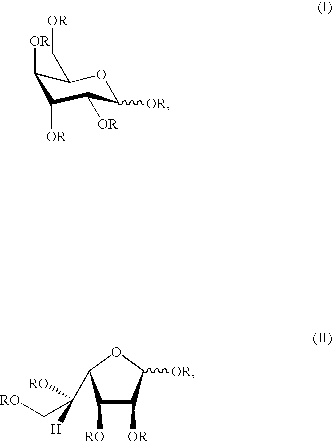
wherein each R is independently H, an alpha-ketoalkanoate group or a C1—C3 acyl and at least one R is an alpha-ketoalkanoate group.

18. The method of claim 7, wherein said alpha-ketoalkanoic acid ester is a dihydroxyacetone ester.

19. The method of claim 7, wherein said alpha-ketoalkanoic acid ester is a thiolester.

20. The method of claim 19, wherein said thiol portion of said thiolester is cysteine or homocysteine.

21. The method of claim 12, wherein said ester of an alpha-ketoalkanoic acid is selected from the group consisting of ethyl pyruvate, propyl pyruvate, carboxymethyl pyruvate, acetoxymethyl pyruvate, carbethoxymethymethyl pyruvate, and ethoxymethyl pyruvate.

22. The method of claim 12, wherein said ester of an alpha-ketoalkanoic acid is selected from the group consisting of ethyl alpha-keto-butyrate, ethyl alpha-keto-pentanoate, ethyl alpha-keto-3-methyl-butyrate, ethyl alpha-keto-4-methyl-pentanoate, and ethyl alpha-keto-hexanoate.

23. The method of claim 1 wherein the subject is administered an effective amount of an amide of an alpha-ketoalkanoic acid.

24. The method of claim 23, wherein said amide of an alpha-ketoalkanoic acid portion is a pyruvamide.

25. The method of claim 23, wherein said amide is an amoni acid amide of an alpha-ketoalkanoic acid.

26. A method of treating acute pancreatitis, said method comprising administering to a subject an effective amount of ethyl pyruvate.

27. The method of claim 26 wherein said acute pancreatitis is acute edematous pancreatitis.

28. The method of claim 26 wherein said acute pancreatitis is acute hemorrhaging pancreatitis.

29. The method of claim 26 wherein said pancreatitis is acute necrotizing pancreatitis.

30. The method of claim 26 wherein said subject is treated prophylactically.

31. The method of claim 26 wherein said ethyl pyruvate is contained in Ringer's Lactate Solution.

32. The method of claim 26 wherein said ethyl pyruvate is contained in a physiologically-acceptable carrier additionally comprising calcium or magnesium.
